(19) United States
(12) Patent Application Publication
(10) Pub. No.: US 2026/0166274 A1
(43) Pub. Date: Jun. 18, 2026

(54) AUTOMATED STORAGE FOR AN ELONGATED SURGICAL TOOL

(71) Applicant: Microbot Medical Ltd., Yokneam Illit (IL)

(72) Inventors: Simon SHARON, Zichron Yaacov (IL); Idan BOADER, Karmiel (IL); Oren COHEN, Kadima (IL)

(73) Assignee: Microbot Medical Ltd., Yokneam Illit (IL)

(21) Appl. No.: 18/711,895

(22) PCT Filed: Nov. 18, 2022
(86) PCT No.: PCT/IL2022/051233

§ 371 Date: May 21, 2024

Related U.S. Application Data
(60) Provisional application No. 63/283,628, filed on Nov. 29, 2021.

(51) Int. Cl. A61M 25/01; A61M 25/09

(52) U.S. Cl. CPC A61M 25/0113; A61M 25/09041

(57) ABSTRACT

An aspect of some embodiments of the invention relates to a storage compartment for one or more elongated surgical tools for an automated robotic device configured to receive and drive movement of elongated surgical tools.

### RELATED APPLICATION/S

This application claims the benefit of priority of U.S. Provisional Ser. No. 63/283,628 filed on 29 Nov. 2021, the contents of which are incorporated herein by reference in their entirety.

### FIELD AND BACKGROUND OF THE INVENTION

The present invention, in some embodiments thereof, relates to an automated storage for elongated surgical tools used in a robotic device for the manipulation of the elongated surgical tools and, more particularly, but not exclusively, to an automated storage for parts used by a robotic device configured to receive and drive movement of elongated surgical tools.

U.S. Pat. No. 8,480,618 to Wenderow et al. discloses: “A robotic catheter system is provided. The robotic catheter system includes a housing and a drive assembly coupled to the housing. The drive assembly is configured to impart movement to a catheter device. The catheter system includes a release structure permitting the drive assembly to be decoupled and removed from the housing without removing the catheter device from a patient.”

### SUMMARY OF THE INVENTION

Following is a non-exclusive list including some examples of embodiments of the invention. The invention also includes embodiments which include fewer than all the features in an example and embodiments using features from multiple examples, also if not expressly listed below.
Example 1. A storage for elongated surgical tools used in a robotic device, the storage comprising a hollowed body sized and shaped to store at least one elongated surgical tool within; said body comprising a proximal end configured to be connected to said robotic device; said connecting allows an internal space of said storage to be in communication to an internal pathway of said robotic device, thereby allowing said elongated surgical tool stored in said storage to be driven back and forth between said storage and said robotic device.
Example 2. The storage according to example 1, further comprising an optional connector for reversibly connecting said hollowed body to said robotic device.
Example 3. The storage according to example 2, wherein said optional connector is an integral part of said storage.
Example 4. The storage according to example 2, wherein said optional connector is provided with said storage and said elongated surgical device.
Example 5. The storage according to any one of examples 1-4, wherein said hollowed body comprises an internal diameter sized and shaped to store one or more liquids in addition to said elongated surgical tool.
Example 6. The storage according to example 3, wherein said internal diameter comprises a range of from about 1 mm to about 6 mm.
Example 7. The storage according to any one of examples 1-6, wherein said hollowed body comprises a spiral form.
Example 8. The storage according to any one of examples 1-7, wherein said hollowed body is configured to store said elongated surgical tool while avoiding bending and/or damaging said elongated surgical tool.
Example 9. The storage according to example 2, wherein said optional connector comprises one or more connecting elements configured to allow connection of storages having different external diameters to said robotic device.
Example 10. The storage according to example 9, wherein said one or more connecting elements comprise one or more concentric steps narrowing in diameter along an inner wall of said optional connector.
Example 11. The storage according to any one of examples 1-10, wherein said elongated surgical tool comprises a guidewire, a microcatheter or both.
Example 12. The storage according to any one of examples 1-11, wherein said storage comprises an original package provided with said elongated surgical tool.
Example 13. The storage according to any one of examples 1-12, wherein said storage comprises a sack/bag.
Example 14. The storage according to any one of examples 1-13, wherein said storage comprises a reel for reeling in and out said elongated surgical tool from said robotic device.
Example 15. The storage according to any one of examples 1-14, further comprising a mechanical support for said storage.
Example 16. The storage according to example 15, wherein said mechanical support is part of said storage.
Example 17. The storage according to example 15, wherein said mechanical support is part of said robotic device.
Example 18. The storage according to any one of examples 1-17, further comprising a bridging element configured to provide geometrical alignment between said proximal end of said storage and said robotic device.
Example 19. The storage according to any one of examples 1-18, wherein said hollowed body is as long as said elongated surgical tool.
Example 20. The storage according to any one of examples 1-19, wherein said body is configured to gravitationally hang from said robotic device and stiff enough to prevent damage and/or allow free movement of said elongated surgical tool.
Example 21. The storage according to example 5, wherein said one or more liquids are one or more of saline, water, oil and lubricant.
Example 22. A method of using a storage for an elongated surgical tool used in a robotic device, the method comprising:
   a. connecting said storage to said robotic device; an internal space of said storage to be in communication to an internal pathway of said robotic device;
   b. feeding an end of said elongated surgical tool into said robotic device;
   c. actuating said surgical device to move said elongated surgical tool.
Example 23. The method according to example 22, further comprising driving said elongated surgical tool back and forth from said storage to said robotic device and vice versa.
Example 24. The method according to example 22 or example 23, further comprising automatically retracting said elongated surgical tool from a location inside a second elongated surgical tool into said storage.
Example 25. The method according to example 24, automatically returning to said location inside said second elongated surgical tool.
Example 26. The method according to any one of examples 22-25, before said connecting, the method further comprises taking out said elongated surgical tool from its original package and inserting said elongated surgical tool into said storage.
Example 27. The method according to any one of examples 22-26, before said connecting, the method further comprises taking out said elongated surgical tool from its original package and inserting said elongated surgical tool into said robotic device before inserting said elongated surgical tool into said storage.
Example 28. The method according to any one of examples 22-27, wherein said connecting comprises connecting an original package of said elongated surgical tool to said robotic device and utilizing said original package as said storage.
Example 29. The method according to any one of examples 22-28, further comprising providing one or more liquids to said storage.
Example 30. The method according to any one of examples 22-29, further comprising maintaining one or more of humidity, cleanness and sterility of said elongated surgical device by said storage.
Example 31. The method according to any one of examples 22-30, further comprising keeping said elongated surgical tool wet during said actuating of said surgical device.
Example 32. The method according to any one of examples 22-31, wherein said feeding is performed before said connecting.
Example 33. The method according to any one of examples 22-32, further comprising continuing providing liquids into said storage during said actuating.
Example 34. The method according to any one of examples 22-33, wherein said feeding is performed before said connecting.
Example 35. A system for manipulation of at least one elongated surgical tool, comprising:
a. a robotic device configured to manipulate said at least one elongated surgical tool;
b. a storage comprising a hollowed body sized and shaped to store said at least one elongated surgical tool within.
Example 36. The system according to example 35, wherein said body comprises a proximal end configured to be connected to said robotic device; said connecting allows an internal space of said storage to be in communication to an inner pathway of said robotic device, thereby allowing said elongated surgical tool stored in said storage to be driven back and forth between said storage and said robotic device.
Example 37. A robotic device for manipulation of at least one elongated surgical tool, comprising:
   a. a housing including walls which define an inner volume containing:
   i. at least one elongate channel for receiving said at least one elongate surgical tool, the channel having at least one first aperture leading into or out from the housing;
   ii. a driving assembly for driving one or both of linear movement and roll movement of said at least one elongate surgical tool, when said at least one elongate surgical tool is received within said channel;
   b. at least one connector in communication with said channel, said connector comprising a branch defining a second aperture located at or externally beyond the walls of said housing, said second aperture being separate from said first aperture of the channel;
   c. a storage comprising a hollowed body sized and shaped to store said at least one elongated surgical tool within; said body comprising a distal end configured to be connected to said first aperture of said channel of said robotic device; said connecting allows an internal space of said storage to be in communication to said channel of said robotic device, thereby allowing said elongated surgical tool stored in said storage to be driven back and forth between said storage and said robotic device.
Example 38. The robotic device according to example 37, further comprising an optional connector for reversibly connecting said hollowed body to said first aperture of said channel of said robotic device.
Example 39. A robotic device for manipulation of at least one elongated surgical tool, comprising:
   a. a housing including walls which define an inner volume containing:
   i. at least one elongate channel for receiving said at least one elongate surgical tool, the channel having at least one first aperture leading into or out from the housing;
   ii. a driving assembly for driving one or both of linear movement and roll movement of said at least one elongate surgical tool, when said at least one elongate surgical tool is received within said channel;
   b. at least one connector in communication with said channel, said connector comprising a branch defining a second aperture located at or externally beyond the walls of said housing, said second aperture being separate from said first aperture of the channel;
   c. a connector for reversibly connecting a storage to said first aperture of said channel of said robotic device.
Example 40. The robotic device according to example 39, wherein said storage comprises a hollowed body sized and shaped to store said at least one elongated surgical tool within; said body comprising a proximal end configured to be connected to said first aperture of said channel of said robotic device; said connecting allows an internal space of said storage to be in communication to said channel of said robotic device, thereby allowing said elongated surgical tool stored in said storage to be driven back and forth between said storage and said robotic device.
Example 41. The robotic device according to example 39, wherein said storage is an original package where said at least one elongated surgical tool is stored.
Example 42. A connector for connecting a storage to a robotic device, comprising:
   a body comprising a body proximal end and a body distal end; said body proximal end configured to receive a distal end of said storage; said body distal end configured to be connected to an aperture in said robotic device.
Example 43. The connector according to example 42, wherein said body comprises one or more concentric steps narrowing in diameter along an inner wall of said connector.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. In the drawings:

FIG. 1 is a schematic block diagram of a robotic device configured for manipulating two or more elongated surgical tools, according to some embodiments of the invention;

FIG. 2 is a schematic representation of an exemplary robotic device for manipulation of two or more elongated surgical tools configured for a telescopic arrangement, according to some embodiments of the invention;

FIG. 3 is a flowchart of an exemplary method for loading elongated surgical tool(s) onto the compact robotic device, according to some embodiments of the invention;

FIG. 4a is a schematic block diagram of a robotic device configured for manipulating two or more elongated surgical tools comprising at least one dedicated storage, according to some embodiments of the invention;

FIG. 4b is a schematic representation of an exemplary robotic device with an exemplary spiral storage, according to some embodiments of the invention;

FIG. 4c is a schematic cross-section representation of an exemplary robotic device comprising a spiral storage showing a guidewire inserted therein, according to some embodiments of the invention;

FIG. 4d is a schematic representation of an exemplary robotic device with an exemplary reversibly removable spiral storage, according to some embodiments of the invention;

FIG. 4e are schematic representations of exemplary adaptors/connectors, according to some embodiments of the invention;

FIG. 4f is a schematic representation of an exemplary injecting device, according to some embodiments of the invention;

FIGS. 4g-4j are schematic representations of exemplary alternative embodiments of the storage, according to some embodiments of the invention;

FIG. 4k, is a schematic representation of an optional diverter, according to some embodiments of the invention;

FIG. 5 is a flowchart of an exemplary method, according to some embodiments of the invention;

FIGS. 6a-i are a schematic diagrams of exemplary driving wheel assemblies for linearly and/or rotationally moving elongate surgical tools, according to some embodiments of the invention; and

FIGS. 7a-c are a flowchart of a method for setting a reference position of an elongate tool in its designated channel of the robotic device, according to some embodiments of the invention and a schematic representation of a tool in a first and a second position, according to some embodiments of the invention.

### DESCRIPTION OF SPECIFIC EMBODIMENTS OF THE INVENTION

The present invention, in some embodiments thereof, relates to an automated storage for a robotic device for the manipulation of elongated surgical tools and, more particularly, but not exclusively, to an automated storage for parts used by a robotic device configured to receive and drive movement of elongated surgical tools.

### Overview

An aspect of some embodiments of the invention relates to a storage compartment for one or more elongated surgical tools for a robotic device configured to receive and drive movement of elongated surgical tools. In some embodiments, the storage is configured to house one or more elongated surgical tools within while safeguarding the integrity of the elongated surgical tools. In some embodiments, storing the one or more elongated surgical tools comprises not damaging and/or bending the elongated surgical tool. In some embodiments, the storage is configured to allow removal and reinsertion of the elongated surgical tool from and to the storage in an easy manner, and thereby into and from the robotic device, for example, by avoiding entanglement of the elongated surgical tool while outside the robotic device. In some embodiments, avoiding entanglement is performed by providing a rigid body to the storage. In some embodiments, avoiding entanglement is performed by providing a flexible body to the storage that is rigid enough to allow protection of the elongated surgical tool. In some embodiments, avoiding entanglement is performed by providing a reel on which the elongated surgical tool is inserted during the operation of the robotic device.

An aspect of some embodiments of the invention relates to a storage compartment for one or more elongated surgical tools for a robotic device configured to receive and drive movement of elongated surgical tools, the storage configured to keep the elongated surgical tool wet during actuation of the robotic device. In some embodiments, the storage comprises a dedicated liquid feeding device, connected to the storage. In some embodiments, the liquid feeding device is an automatic liquid feeding device configured to sense one or more levels of liquids inside the storage and further configured to feed additional liquids when necessary and/or when a certain amount and/or level of liquids inside the storage is reached.

An aspect of some embodiments of the invention relates to a robotic device comprising an optional connector sized and shaped to connect with a storage compartment fit for storing an elongated surgical tool. In some embodiments, the optional connector is located at an entry port to an inner pathway of the robotic device. In some embodiments, the inner pathway of the robotic device is defined between an entry port and an exit port found on opposite sides of the robotic device housing. In some embodiments, the inner pathway is sized and shaped for allowing passage of at least a portion of the elongated surgical tool. In some embodiments, the inner pathway comprises an actuating assembly, e.g. driving wheels and motors, for driving the elongated surgical tool linearly across the entry port, thereby advancing the elongated surgical tool from the storage compartment and into the patient's body, or retracting the elongated tool from the patient body into the storage compartment. In some embodiments, when there is no optional connector, the storage compartment can be positioned near the robotic device, optionally attached to the bed where the patient is located during the procedure. In some embodiments, the storage compartment can be mounted on the bed where the patient is located during the procedure and the storage compartment is indirectly connected to the robotic device optionally using an additional accessory, either with or without the optional connector.

An aspect of some embodiments of the invention relates to a robotic device comprising an optional diverter configured to direct an elongated tool exiting from the proximal side of the robotic device to a desired direction, where potentially will not disturb either the user or the patient. In some embodiments, the diverter is configured to be manipulated and/or bended and also configured to remain in the manipulated and/or bended position.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Robotic Device Configured to Receive and Drive Movement of Elongated Surgical Tools

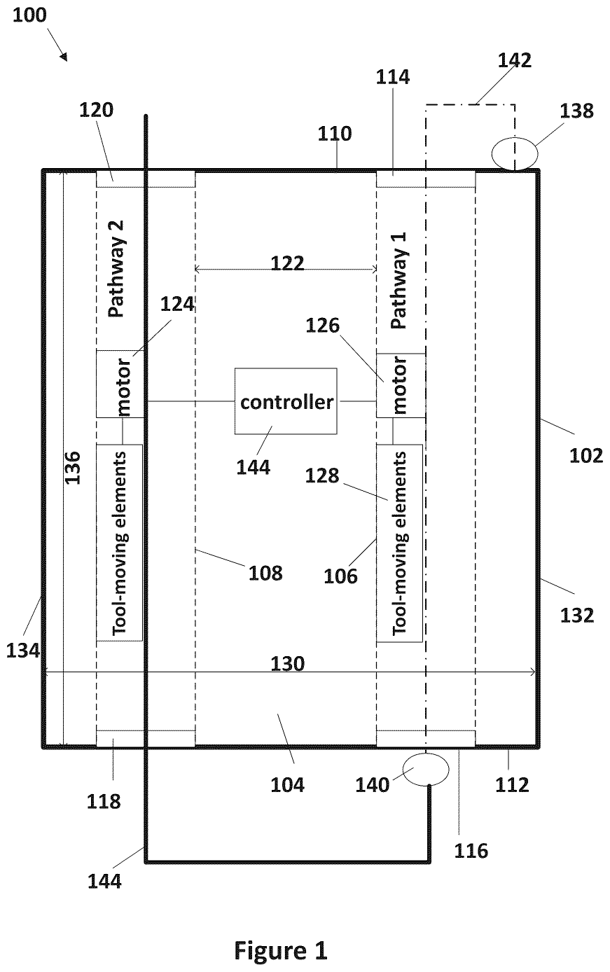

Referring now to FIG. 1, showing a schematic block diagram of a robotic device configured for manipulating two or more elongated surgical tools, according to some embodiments of the invention.

In some embodiments, walls of a housing 102 of the robotic device 100 define an inner volume 104 in which at least two distinct pathways (channels) such as 106, 108 for the elongated surgical tools are defined. In some embodiments, the pathways extend across the inner volume, for example, between two opposing walls of the housing, such as wall 110 and wall 112. Optionally, the housing is shaped in an elongated form, for example having a substantially rectangular cross section profile, and the pathways extend along the length of the housing.

In some embodiments, each of the pathways extends between an entry aperture formed at the wall of the housing, and an exit aperture formed at an opposite wall of the housing. In the example shown, pathway 106 extends between entry aperture 114 formed at wall 110 and an exit aperture 116 formed at wall 112; and pathway 108 extends between an entry aperture 118 formed at wall 112 and an exit aperture 120 formed at wall 110.

In some embodiments, an aperture formed in a wall of the housing is shaped and/or sized according to the surgical tool that is passed through it. For example, a rounded (e.g. circular) aperture is sized for fitting a cylindrical tool, such as a guidewire or microcatheter, where the aperture diameter is optionally no more than 5%, 10%, 25% or intermediate, higher or smaller percentage larger than a diameter of the tool. In some embodiments, an aperture is sized for more than one tool to be passed through. Optionally, the aperture profile is oval (e.g. ellipsoid), rectangular, slot shaped and/or other. In some embodiments, a single elongated slot serves as an aperture for both inner pathways.

In some embodiments, a single tool passes through an entry aperture into the inner volume of the housing, and exits the housing through a respective exit aperture. Alternatively or additionally, in some embodiments, a plurality of tools telescopically arranged (e.g. 2 tools, such as a guidewire provided within the inner lumen of a microcatheter) pass together through the same entry aperture and exit the housing together through a respective exit aperture. Thus, in such an example, a first tool passes through a first inner pathway, exits the housing into the lumen of a second tool, and the telescopic assembly of both tools passes through a second inner pathway. In some embodiments, the telescopic arrangement of the tools occurs outside of the housing, after both tools have passed through their inner pathways, for example, in the case of a rapid exchange catheter which can be interfaced with the guidewire after each of the guidewire and the rapid exchange catheter have passed independently through their respective actuation assemblies located in the inner pathways.

In some embodiments, the pathways extend in a similar plane, for example, a similar horizontal plane, a similar vertical plane, a similar plane extending diagonally between the walls of the housing. In some embodiments, the pathways extend along parallel axes. A distance 122 between the parallel axes may range, for example, between 3-12 cm, 2-10 cm, 5-9 cm or intermediate, longer or shorter distance.

Alternatively, in some embodiments, the pathways are not parallel, for example, one pathway extends directly between opposite walls while another takes a diagonal or other indirect route.

In some embodiments, except for the aperture locations, the housing is sealed. Optionally, the housing includes a removable or moveable cover or lid. In some embodiments, the housing is open at least in part, for example, shaped as a box with no top face.

In some embodiments, all components which engage the tool to manipulate it and/or to drive its movement are fully encased inside the inner volume of the housing and at least some of these components are positioned along the pathway defined for the tool. In some embodiments, these components include an actuation assembly, for example the tool-moving elements (e.g. driving wheels).

In some embodiments, as shown, a plurality of motors 124, 126 are configured to drive the actuation assemblies, for example configured to drive tool-moving elements 128 (e.g. wheels) of each assembly. In some embodiments, the motor and the tool moving elements are positioned along the pathway defined for the tool. In some embodiments, the actuation assemblies of the two (or more) pathways are aligned side-by-side. A potential advantage of the actuation assemblies being aligned side-by side may include allowing for a short or minimal distance 130 (optionally being the device width or height) between opposing walls 132, 134. In an example, distance 130 is smaller than 15 cm, 12 cm, 10 cm or intermediate, longer or shorter distance.

In some embodiments, the actuation assemblies of the two or more pathways have a similar axial extent (or do not extend beyond a certain axial extent). A potential advantage of the actuation assemblies being positioned relative to each other and/or sized such that they do not extend beyond a certain axial extent may include that a distance 136 between walls 110 and 112 (optionally being the device length) may be kept to a minimal axial extent needed to contain the movement driving components. In an example, distance 136 is smaller than 10 cm, 7 cm, 12 cm or intermediate, longer or shorter distance. In some embodiments, the plurality of motors 124, 126 are also positioned within the axial extent of the actuation assemblies, and in proximity to the actuation assemblies, to facilitate the compact design of the device. In some embodiments, the ability to position the motor(s) in close proximity to the actuation assemblies and potentially in contact with at least a portion of the actuation assemblies is provided, for example, due to that no barriers (e.g. sterile protection or shield) are needed between the actuation assembly, the motor(s), and the surgical tool being manipulated.

In some embodiments, the actuation assemblies of the two or more pathways are positioned within the same, shared inner volume defined by the walls of the housing. In some embodiments, no barriers (e.g. inner walls, shields, drapes, and the like) exist between the movement driving components of the two or more pathways. In some embodiments, no barriers (e.g. inner walls, shields, drapes, and the like) exist between the actuation assemblies and the tools that are being manipulated by them.

Alternatively, in some embodiments, a partial partition or barrier are provided. For example, the device housing may include an inner wall or protrusion which do not fully block the inner volume, leaving at least some regions of the pathways in communication with each other.

In some embodiments, an actuation assembly of an inner pathway (e.g. an actuation assembly that includes a shaft in which a tool is received and/or wheels which drive linear movement of the tool) is exposed to an actuation assembly of a different inner pathway, for example an adjacent pathway.

In some embodiments, actuation assemblies of a plurality of pathways are arranged and held with respect to each other on a chassis. Optionally, the chassis is exposed and open to its surroundings, for example, no housing is provided.

In some embodiments, an actuation assembly of a pathway at least partially restrict movement of the tool within the inner pathway, for example, restricting lateral movement of a tool received within the pathway. For example, movement of the tool out of notional limits defined by the elongated pathway is restricted. In some embodiments, the tool is channeled through the pathway, for example, received within a slot of an elongated shaft. Alternatively or additionally, the pathway is defined by a path generated between a plurality of pairs of opposing wheels.

In some embodiments, in addition to extending through the pathway, a tool engages the device at one or more additional fixation locations (also referred to herein as “securing points”, “engagement points”). In some embodiments, a fixation location comprises a holder (such as 138, 140) located outside the housing, inside the housing, or partially inside the housing and partially outside the housing. In some embodiments, a fixation location couples a tool to the housing and/or to one or more other tools. For example, at fixation location 140 a first elongated surgical tool 142 which extends through pathway 106 (e.g. a guidewire) enters an inner lumen of a second elongated surgical tool 144 (e.g. a microcatheter), which is coupled to the housing at fixation location 140. In some embodiments, a proximal end of tool 142 is coupled to the housing at fixation location 138.

In some embodiments, fixation location 138 is shaped and configured to accommodate a proximal handle of tool 142, for example, a handle that manipulates the distal portion of the tool in terms of bend and/or stiffness. In some embodiments, an additional motor (not shown) is configured for rotating tool 142 through two locations, one of which is the handle of the tool (for example at fixation location 138) and the other is a region more distal of the tool. For example, a motor configured for rotating tool 142 by rotating an actuation assembly which is associated with a portion of the tool 142, is also operably connected to the handle of the tool, optionally through a gear system. As such, the motor is configured for rotating the tool simultaneously from these two distinct locations. An advantage for commencing roll movement by the same motor in two different locations along the tool may include enhancing the torque applied on the tool and eliminating the risk of slippage of the tool in its gripping locations found in the actuation assembly.

In some embodiments, a fixation location of a tool with the housing (such as 138) and an entry aperture leading the tool into the inner volume (such as 114) are located on a same wall of the housing, so that a section of the tool that is found outside the housing forms a curve, for example, a U-shaped curve. In some embodiments, the extent of the U-curve is dynamically adjustable. Optionally, linearly moving the tool (such as via the tool-moving elements, e.g. wheels) changes the extent of the U-curve relative to the external side of the wall of the housing.

In some embodiments, the curve is defined along a path which extends from and to the same wall of the device housing.

In the example shown, the housing comprises sharp corners and straight edge walls, but other configurations are also contemplated, including, for example, rounded corners, curved walls, and the like.

In some embodiments, actuation of the actuation assembly (e.g. via a motor) of each of the pathways is controlled by a controller 144. In some embodiments, components of each pathway are controlled independently, yet in a synchronized manner.

In some embodiments, controller 144 is controlled remotely by an external device, for example by a remote control device such as described herein.

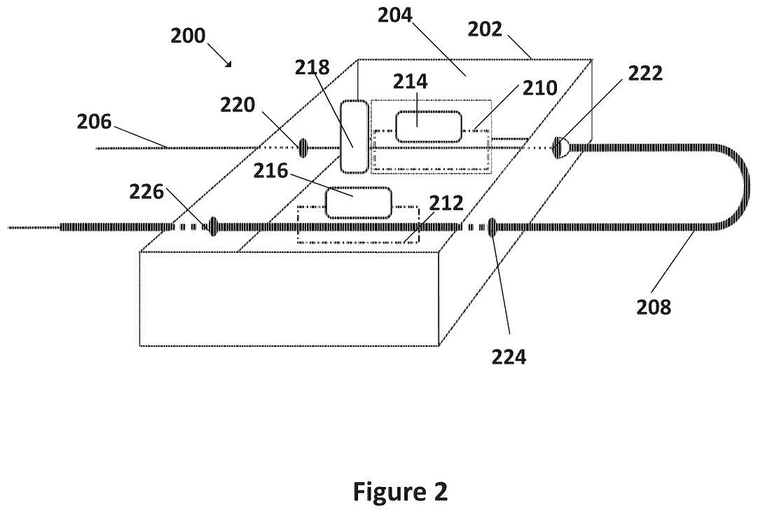

Referring now to FIG. 2, showing a schematic representation of an exemplary robotic device for manipulation of two or more elongated surgical tools configured for a telescopic arrangement, such as in a non-limiting manner a guidewire and a microcatheter, the first elongated tool extending at least in part within the lumen of the second elongated tool, according to some embodiments of the invention.

In some embodiments, robotic device 200 comprises a housing 202 comprised of a plurality of walls which form an inner volume 204 between them. In some embodiments, two or more inner pathways extend inside the inner volume, such that tools 206, 208 received and operated by the device 200 extend, at least in part, along the inner pathways.

In some embodiments, each of the inner pathways includes an actuation assembly positioned at a position of the pathway, for example, axially extending along at least a portion of the pathway. In some embodiments, an actuation assembly, such as 210, 212, is configured for linearly moving the tool, for example, one or more sets of wheels configured to advance and/or retract the tool. Alternatively or additionally, an actuation assembly, such as 210, is configured for moving the tool in a roll manner, for example by rotating a set of wheels gripping the tool therebetween.

In some embodiments, actuation assemblies are operably coupled to a plurality of motors, for example motors 214, 216, 218. In some embodiments, the motors are configured for operating the actuation assemblies to generate linear movement of the tools received therein. Alternatively or additionally, the motors are configured to generate a roll movement of the received tool, optionally by generating a roll movement of the tool's associated actuation assembly as a whole. For example, motor 218 is operably connected to linear movement mechanism 212, optionally via a gear system, and is configured to rotate linear movement mechanism 212 together with motor 214, thereby rolling tool 206 which is gripped within linear movement mechanism 212. A potential advantage for rotating the entire linear movement mechanism along with the tool is a simplification of the associated gear system, and the enablement of simultaneous operation of linear and roll movement together. Rolling of motor 214 together with the linear movement mechanism 210 is enabled, in some embodiments, due to that no sterile barrier exists between the motors and the actuation assemblies.

In the example shown, a first elongated surgical tool 206 (e.g. a guidewire) extends along a first inner pathway, for example between an entry aperture 220 into the housing and an exit aperture 222 from the housing.

In some embodiments, linear movement of tool 206 is driven by motor 214, and roll of tool 206 is driven by motor 218, both located and configured at a position of the inner pathway (e.g. along a notional axis defined by the pathway across the inner volume).

In some embodiments, at the exit aperture 212 of tool 206 from the housing, the tool 206 is telescopically received within a lumen of a second elongated surgical tool 208, for example, a microcatheter. Tool 208, in turn, enters the housing at an entry aperture 224 and extends along a second inner pathway to an exit aperture 226, with tool 206 extending inside it.

In some embodiments, linear movement of the tool 208 is driven by actuation assembly 212.

In some embodiments, the actuation mechanism(s) and the plurality of motors all share the same inner volume, with no barrier or other physical separation therebetween.

### Exemplary Method of Loading a Compact Robotic Device With Elongated Surgical Tools

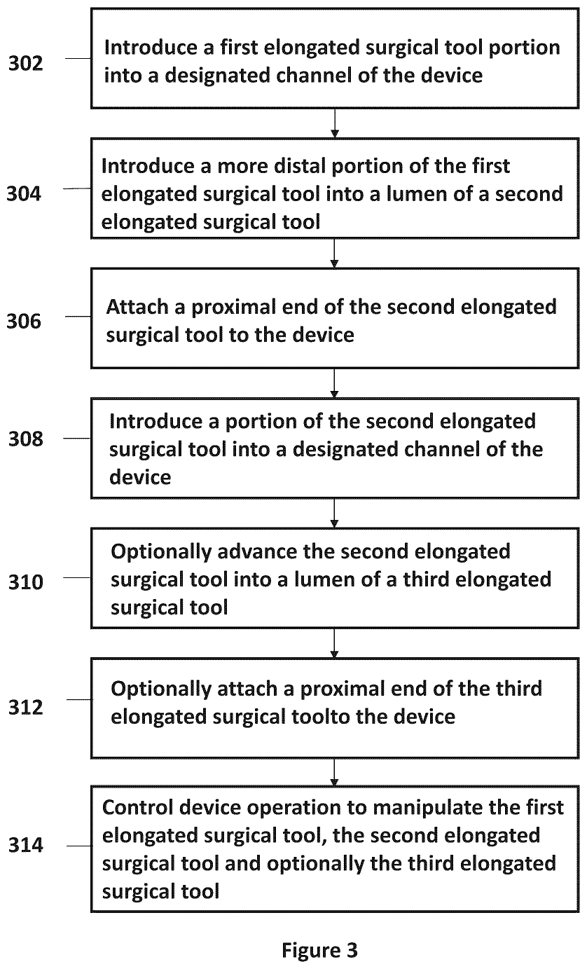

Referring now to FIG. 3, showing a flowchart of an exemplary method for loading elongated surgical tool(s) onto the compact robotic device, according to some embodiments of the invention.

In some embodiments, prior to performing a surgical procedure, the compact robotic device is loaded with one or more elongated surgical tools, which are then manipulated by the device.

The following method is an example for loading the device. It is noted that the steps may be carried out manually (e.g. by a physician, surgeon, nurse or other clinical personnel) or, in some embodiments, automatically.

In some embodiments, a portion of a first elongated surgical tool is introduced into a designated channel, e.g. an inner pathway, of the device 302, for example, a proximal portion of the guidewire, optionally by leading with the proximal end of the elongated surgical tool. In some embodiments, a more distal portion of the first elongated surgical tool, optionally including the first elongated surgical tool distal end, is introduced into a lumen of a second elongated surgical tool 304, for example a microcatheter. The second elongated surgical tool proximal end is then optionally coupled to the device 306, for example at a luer disposed on the device housing, or via another suitable connector. Then, a portion of the second elongated surgical tool is introduced into a designated channel of the device 308. In some embodiments, the second elongated surgical tool forms a curve outside the device housing, for example between a connection of the microcatheter proximal end to the luer and the entry aperture of a more distal portion of the second elongated surgical tool into the designated channel. Alternatively, the first elongated surgical tool is introduced into the second surgical tool only in the patient's body, for example, in rapid exchange systems.

In some embodiments, when a third elongated surgical tool, for example a guiding catheter, is used, the second elongated surgical tool (optionally including the first elongated surgical tool threaded therein) is advanced into a lumen of the guiding catheter 310. Optionally, the third elongated surgical tool is introduced to the patient body prior to advancing the tools into its lumen. In some embodiments, the third elongated surgical tool proximal end is then attached to the device 312 for example at a luer disposed on the device housing, or via another suitable connector.

In some embodiments, loading of the device involves introducing the first elongated surgical tool first into contact with the device assemblies, and then introducing the additional tools (e.g. second elongated surgical tool and optionally then a third elongated surgical tool) using the first elongated surgical tool as a backbone for the telescopic arrangement of all tools. In some embodiments, the first elongated surgical tool serves as an introducer which is introduced together with the additional tools into their designated channel inside the robotic system. In some embodiments, as detailed in the example of FIG. 1, the first elongated surgical tool is introduced into its designated channel first and is then used to guide additional tools into their channels. Alternatively, the first elongated surgical tool is first used to introduce the additional tools using its distal end, and only then it is threaded through its proximal end into its own designated space.

In some embodiments, a user controls manipulation of the tools loaded onto the device 314, for example by controlling linear movement and/or rotation of the tools driven by the device assemblies. In some embodiments, control is performed remotely, for example via a remote control, console or the like.

It is noted that a loading method as described is provided only as an example, and that the described steps may be carried out in a different order, and/or that different steps will be performed. In some embodiments, some of the steps are carried out manually and/or are aided by a user, while some of the steps (e.g. advancing, retracting and/or rolling a tool) into a desired position and/or orientation are carried out automatically by the device.

### Exemplary Storage for an Elongated Surgical Tool

In some embodiments, the robotic device configured to receive and drive movement of elongated surgical tools comprises a dedicated storage for elongated surgical tools, for example a guidewire, configured to store and/or kept wet the elongated surgical tool during the medical procedure.

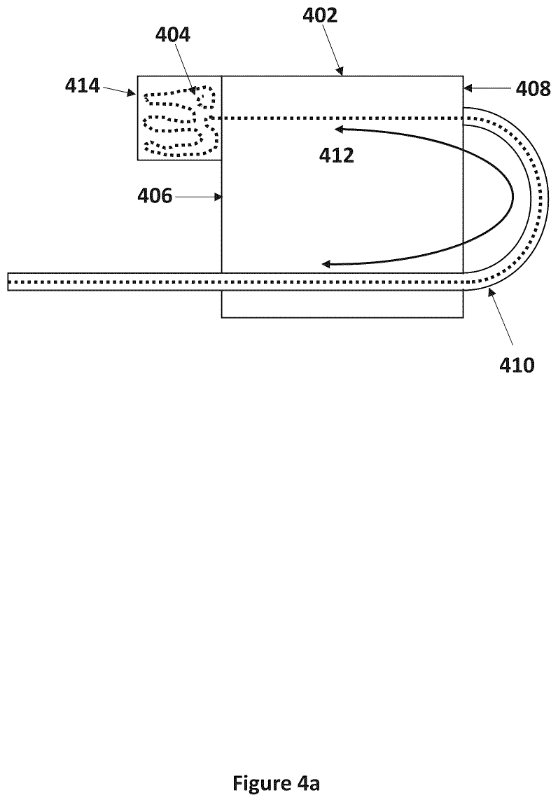

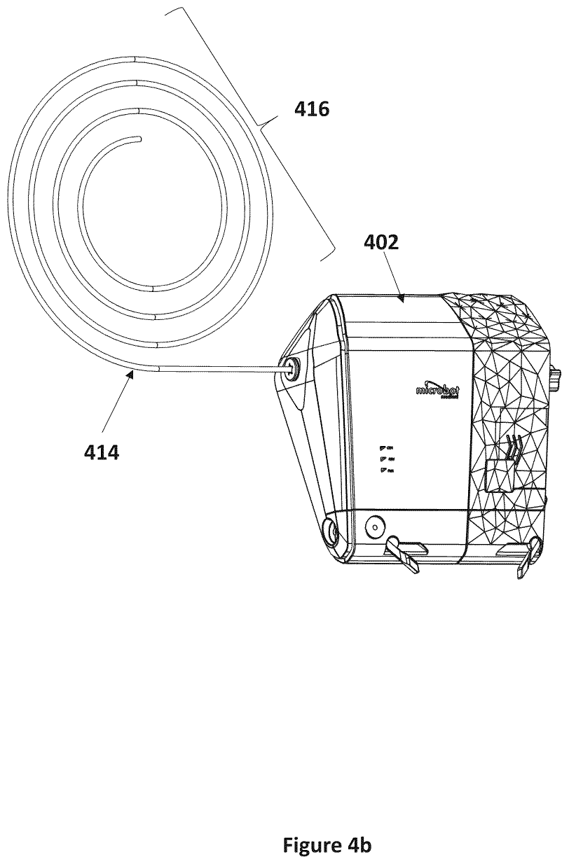

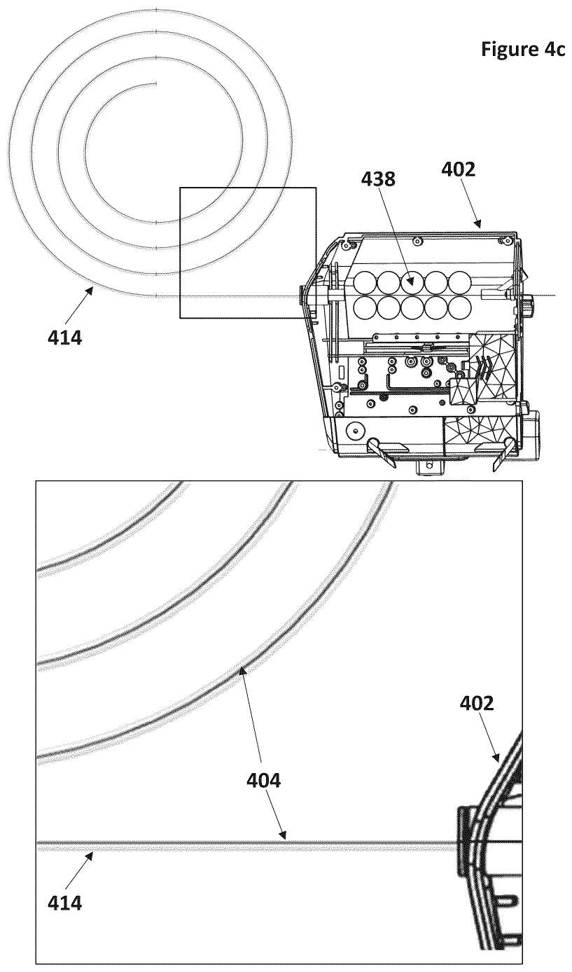

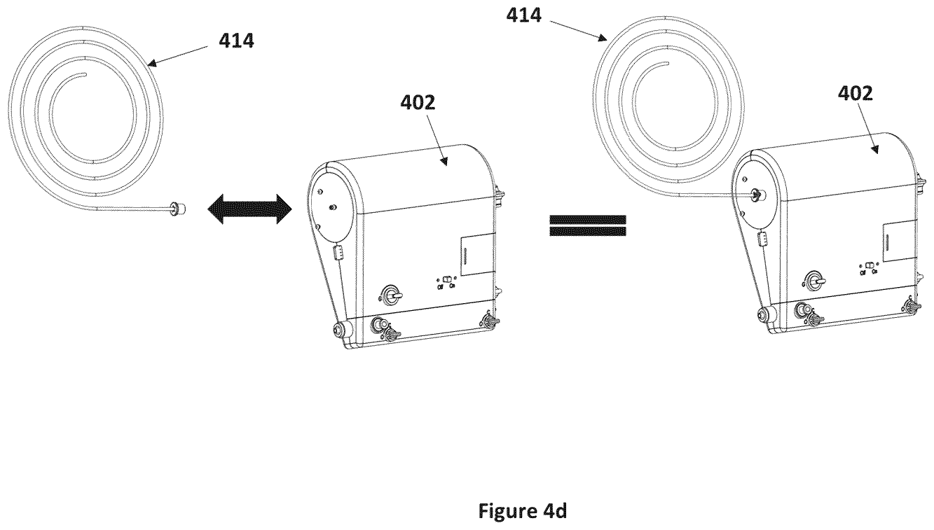

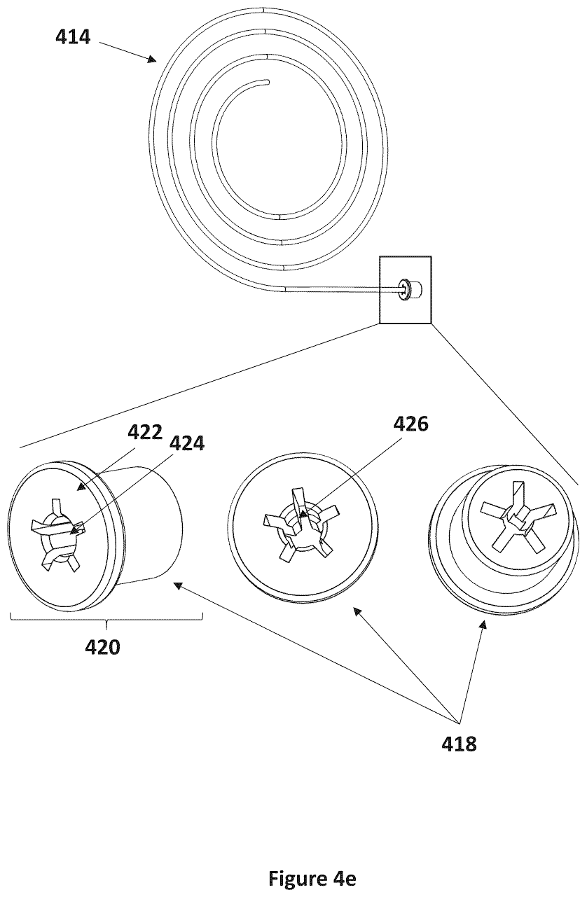

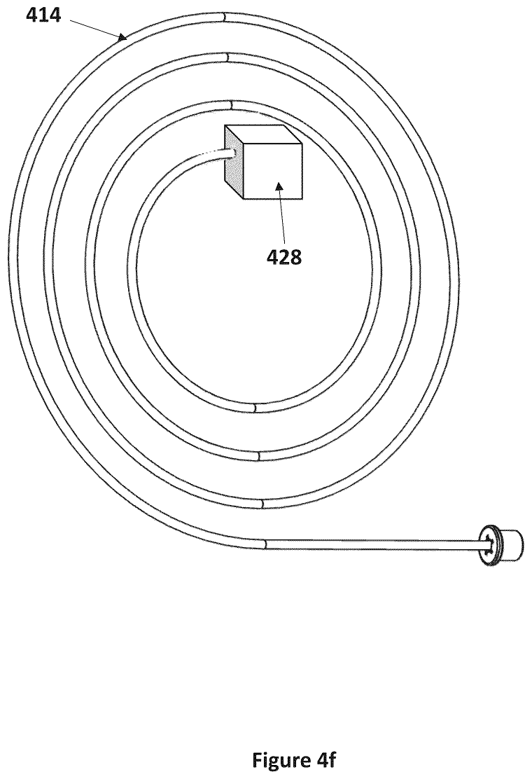

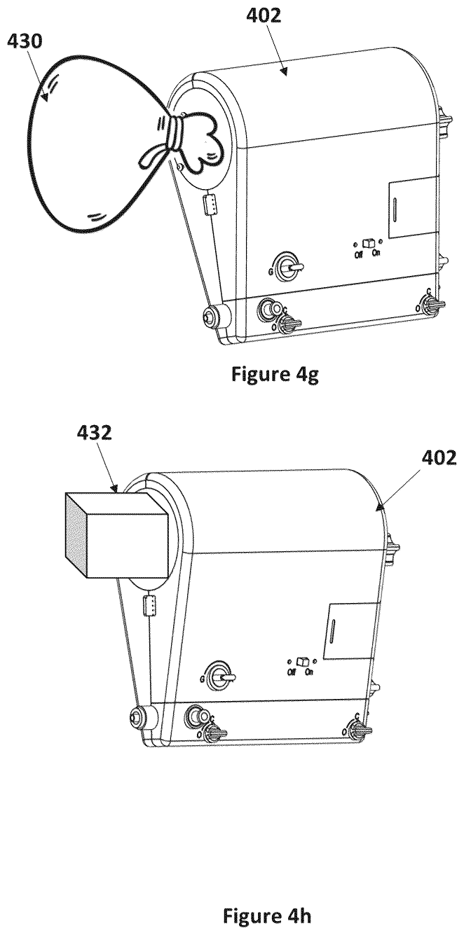

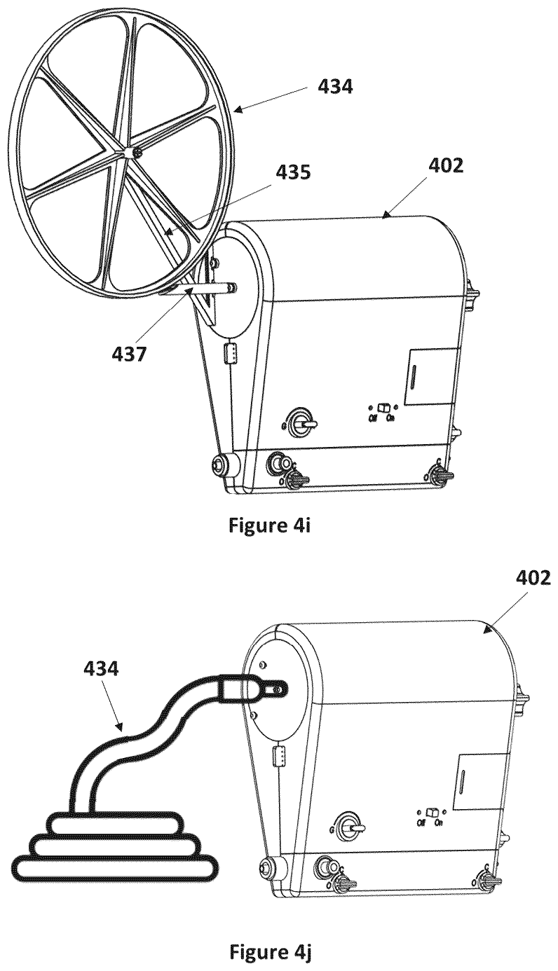

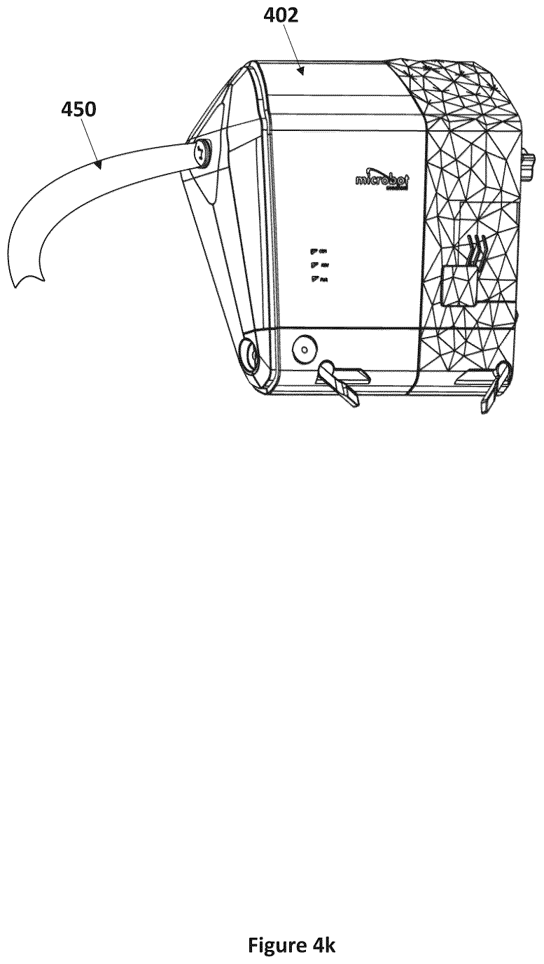

Referring now to FIG. 4a, showing a schematic block diagram of a robotic device configured for manipulating two or more elongated surgical tools comprising at least one dedicated storage, according to some embodiments of the invention. In some embodiments, the device 402 is as disclosed above, and is configured to receive an elongated surgical tool, for example a guidewire 404, from one side of the device for example from side 406. In the following paragraphs a “guidewire” will be used to explain the exemplary embodiments. It should be understood that other elongated surgical tools can be used instead, and also those are part of the scope of the invention. In some embodiments, the guidewire 404 runs inside the device 402, exiting on the opposite side 408, while immediately entering a second elongated surgical tool, for example a microcatheter 410. In some embodiments, as disclosed above, there are a plurality of internal mechanisms, not shown in FIG. 4a but explained in relation to FIGS. 1 and 2, which allow the linear movement of the guidewire 404 forwards and backwards, following the schematic arrow 412, into and out of the microcatheter 410. In some embodiments, the device 402 comprises a dedicated storage 414 for the guidewire 404, as shown for example in FIG. 4a.

In some embodiments, the storage 414 is characterized by a being an elongated tube comprising a level of rigidity, which allows the containment of the guidewire while conserving the form of the container and optionally the liquids therein. In some embodiments, the storage 414 comprises a spiral form 416, as shown for example in FIG. 4b (same numbers were used for same parts). In some embodiments, the storage 414 is characterized by having a form other than a spiral form, see below for exemplary non-spiral storage embodiments. In some embodiments, a potential advantage of having a spiral form is that it potentially keeps the integrity of the guidewire. In some embodiments, another potential advantage of having a spiral form is that it potentially reduces the internal area of the storage, which directly reduces the quantity of liquid required for maintaining the guidewire wet during the procedure (see below).

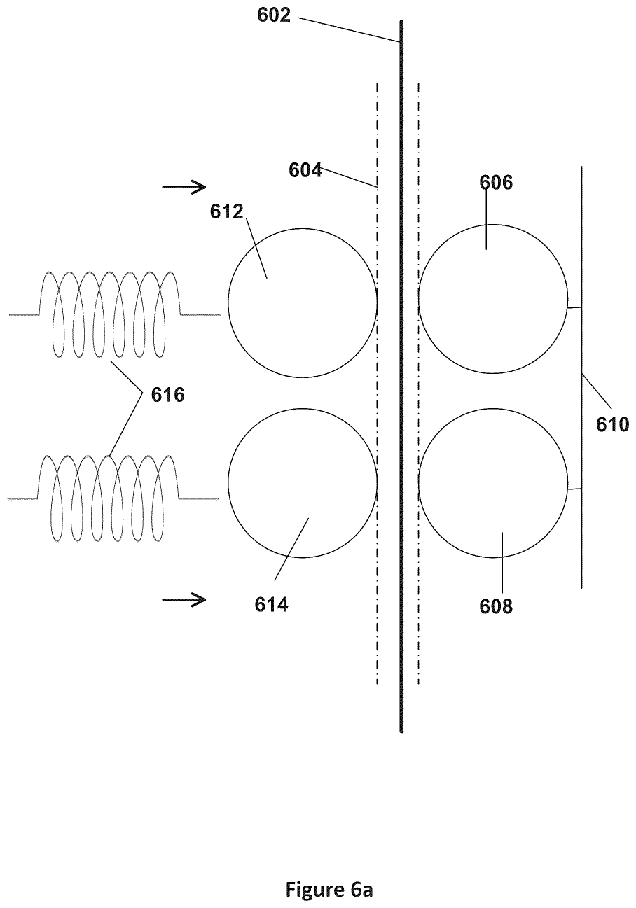

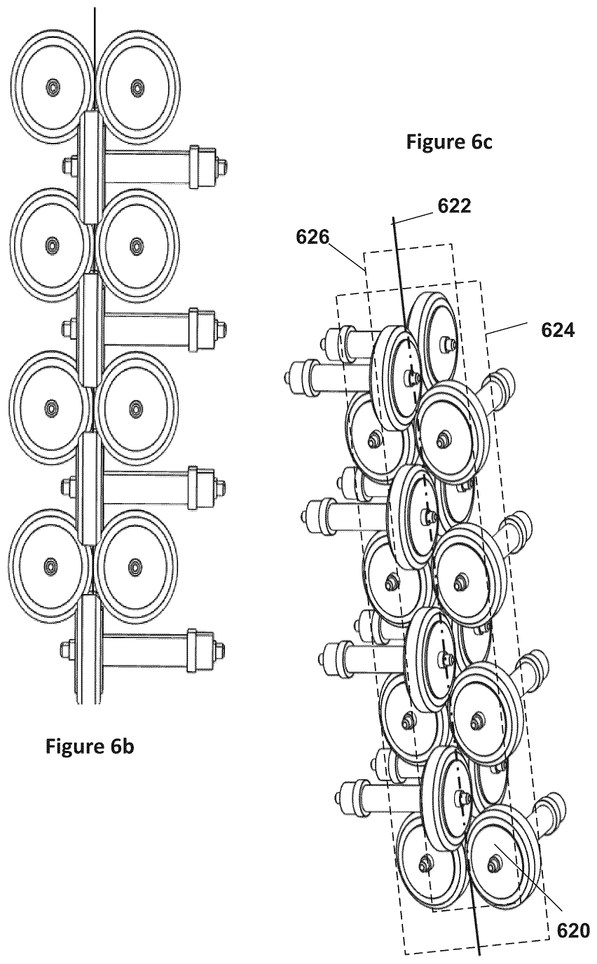

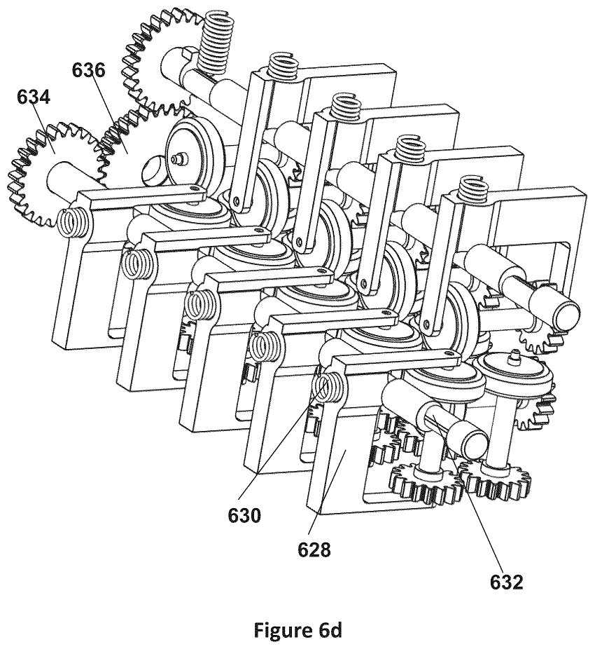

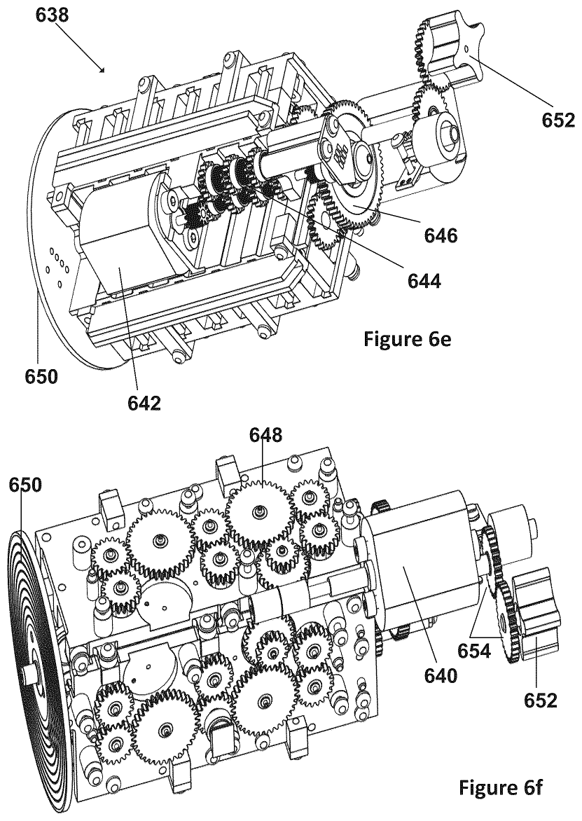

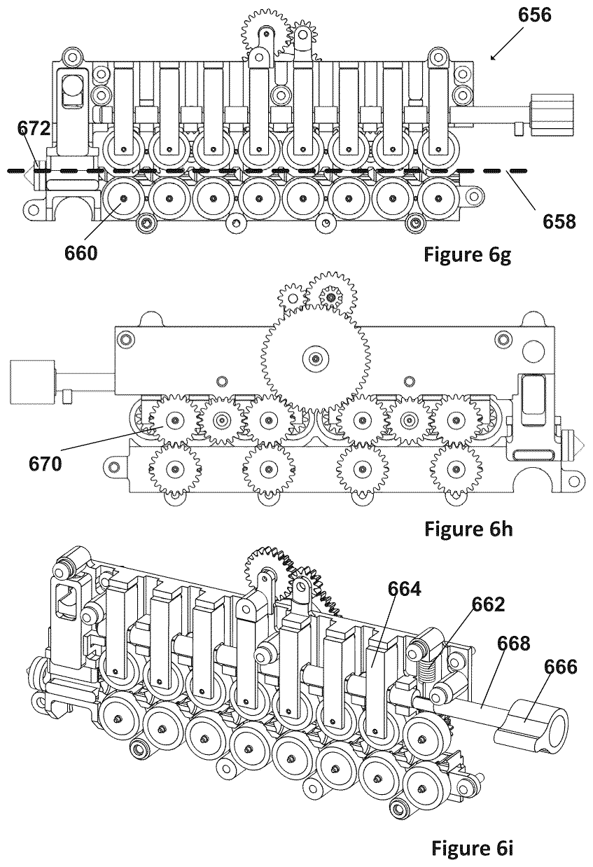

In some embodiments, the spiral storage 412 comprises an internal diameter slightly bigger than that of the external diameter of the guidewire being used. For example, the internal diameter of the storage is from about 0.1 millimeter (mm) to about 3 mm bigger than the external diameter of the guidewire. Optionally, from about 0.01 mm to about 5 mm bigger than the external diameter of the guidewire. Optionally, from about 0.001 mm to about 10 mm bigger than the external diameter of the guidewire. In some embodiments, the internal diameter is from about 1 mm to about 6 mm. FIG. 4 c shows a schematic cross-section representation of a device 402 comprising a spiral storage 414 showing a guidewire 404 inserted therein, according to some embodiments of the invention. In some embodiments, also shown in FIG. 4c, the device 402 comprises a driving wheel assembly 438 comprising a plurality of driving wheels, which will be further explained below in FIGS. 6a-i. In some embodiments, the storage 414 is reversibly attached to the device 402 at the surface at the location of the entry aperture (see for example 220 in FIG. 2) for the guidewire, as shown for example in FIG. 4d. In some embodiments, the storage is a dedicated part of the device 402, and the user is required to take out the guidewire from its original package and insert it in the storage part. In some embodiments, the original package in which the guidewire is provided by the manufacturer is used as the storage part in the device 402.

In some embodiments, a dedicated additional inserter being less pliant than the elongated surgical tool and configured to be attached to the distal end of the elongated surgical tool, is used to facilitate the insertion and/or feeding of the elongated surgical tool into the device. In some embodiments, a potential advantage of using the inserter is that, since the elongated surgical tool may comprise a very pliant distal end, an addition of an inserter being less pliant can potentially ease the insertion of the elongated surgical tool into the device.

Referring now to FIG. 4e, showing schematic representations of exemplary adaptors/connectors 418, according to some embodiments of the invention. In some embodiments, the device 402 further comprises an optional dedicated adaptor/connector 418 configured to be inserted at the entry aperture (see for example 220 in FIG. 2) for the guidewire 404 in the device 402, as shown for example in FIG. 4e. In some embodiments, the optional adaptor/connector comprises a body 420 comprising one or more of a head 422, being wider than the body, and an orifice 424, configured to receive a storage 414. In some embodiments, the orifice 424 comprises one or more steps 426 and or indentations configured to allow the connection of storages having different external diameters, optionally due to guidewires having different diameters, to the device 402. In some embodiments, the steps are one or more concentric steps narrowing in diameter along an inner wall of the optional adaptor/connector.

In some embodiments, the inner wall of the optional connector further comprises a cleaner for the elongated surgical tool configured to clean the elongated surgical tool when passing through it, either back or forth from the storage or from the device.

In some embodiments, when there is no optional connector, the storage compartment can be positioned near the robotic device, optionally attached to the bed where the patient is located during the procedure. In some embodiments, the storage compartment can be mounted on the bed where the patient is located during the procedure and the storage compartment is indirectly connected to the robotic device optionally using an additional accessory, either with or without the optional connector.

### Exemplary Provisions of Liquids Into the Storage

In some embodiments, as mentioned above, the storage is configured to store, in addition to the elongated surgical tool, liquids to keep the elongated surgical tool wet during the procedure. In some embodiments, the liquids are inserted by a user directly into the original package of the elongated surgical tool, which will be also used as the storage of the device. In some embodiments, liquids are inserted from a distal end of the package and/or storage and the liquids remain inside due to capillary forces.

In some embodiments, when an original package is not used, the user takes out the elongated surgical tool out of the original package and inserts it either directly into the robotic device and then, by means of the internal mechanisms of the device, into the storage device, which comprises liquids. Alternatively, the user takes out the elongated surgical tool out of the original package and inserts it directly into the storage device which comprises liquids therein, and introduces the tool from its distal end into the robotic device, optionally by using a supporter.

In some embodiments, when necessary, a user can manually add more fluids into the storage, if and when necessary.

In some embodiments, a dedicated automatic liquid injection device is used.

Referring now to FIG. 4f, showing a schematic representation of an exemplary injecting device, according to some embodiments of the invention. In some embodiments, liquids are inserted inside the storage 414 in order to keep the guidewire wet during the procedure. In some embodiments, liquids can be one or more of saline, water, oil and lubricants. In some embodiments, maintaining the guidewire wet is necessary for the insertion, extraction and re-insertion of the guidewire in the microcatheter during procedures. In some embodiments, the storage comprises a dedicated injecting device 428, configured to provide liquids into the internal space of the storage 414 as necessary. In some embodiments, the injecting device 428 comprises a passive mechanism of injection. For example, the injecting device 428 utilizes negative pressure to provide the necessary liquids, for example, as liquids leave the interior of the storage, new liquids are pulled from the injecting device 428 into the storage 414. In some embodiments, the injecting device 428 comprises an active mechanism of injection. For example, the injecting device 428 comprises a motor, a sensor, an energy source and circuitry. In some embodiments, the sensor is configured to monitor the liquid levels inside the storage 414. In some embodiments, once the levels of the liquids inside the storage 414 arrive at a certain level, optionally predetermined levels of liquids, the motor is activated and new liquids are injected inside the container 414. In some embodiments, circuitry is configured to control the components of the active mechanisms of the injecting device 428. In some embodiments, the energy source can be one or more of a battery, a rechargeable battery and/or a cable to be plugged into the electricity.

Referring now to FIGS. 4g-j, showing schematic representations of exemplary alternative embodiments of the storage, according to some embodiments of the invention. In some embodiments, as mentioned above, the storage 414 comprises a form other than a spiral form, for example, the storage 414 can be a sack/bag 418 attached to the device 402, as shown for example in FIG. 4g. In some embodiments, the sack can be soft, semi-rigid or rigid. In some embodiments, the storage 414 is characterized by having a form of a box 420 that is attached to the device 402, as shown for example in FIG. 4h. In some embodiments, the storage 414 is characterized by having a form of a reel 434 that is attached to the device 402, as shown for example in FIG. 4i. In some embodiments, the reel comprises an internal storing area where the reeled-in/out guidewire is kept, and the storing area optionally comprises liquids for keeping the guidewire wet during the procedure. In some embodiments, the storage and/or the device comprises a mechanical support 435 for the storage, as shown for example in FIG. 4i. In some embodiments, the mechanical support 435 is part of the storage. In some embodiments, the mechanical support 435 is part of the device. In some embodiments, the device comprises a bridging element 437 configured to provide geometrical alignment between the device and the storage. In some embodiments, the storage 414 is characterized by having a form of a lose tube, similar to a hose 436 that is attached to the device 402, as shown for example in FIG. 4j. In some embodiments, the tube is flexible so it can “hang” from the device, but is stiff enough to safeguard the integrity of the guidewire within. In some embodiments, the proximal end of the tube is connected to the device, while the distal end of the tube can be closed or can be configured to allow connection to a liquid providing device for keeping the guidewire wet during the procedure.

### Exemplary Potential Advantages of the Storage

In some embodiments, as disclosed above, the device is used, for example, to manipulate elongated surgical tools. For example, guidewires that are used concomitantly with microcatheters. It is known that during medical procedures involving guide catheters, microcatheters and/or guidewires, it is necessary to insert and retract the guidewire several times. For example, when trying to reach the desired location inside the body of the patient, the user may require to retract the guidewire from the microcatheter, insert contrast material to visualize the path, and then re-insert the guidewire into the microcatheter to continue the procedure to reach the desired location. In this example, these retract/re-insert steps may be repeated several times during the procedure. In relation to this, it is also know that when the guidewire is extracted, the “loose” guidewire outside the body needs to be kept contained to keep it sterile and additionally, the guidewire needs to be kept wet to allow a smooth re-insertion into the microcatheter. Today's solution is to use buckets comprising a type of liquid, where the “loose” guidewire is kept while waiting to be re-inserted.

In some embodiments, potential advantages of the storage disclosed herein are one or more of:
1. Potentially keeping the integrity of the guidewire: in some embodiments, the storage potentially allows the insertion and extraction of the guidewire to and from the storage without causing damage and/or bends in the guidewire.
2. Potentially keeping the guidewire in a state feasible for reinsertion into a microcatheter: in some embodiments, the storage potentially provides an environment to the guidewire that keeps it feasible for reinsertion into a microcatheter. In some embodiments, this is done by keeping the guidewire wet, which is performed by keeping the guidewire in a wet environment while being stored in the storage.
3. Potentially keeping the guidewire available for reinsertion: in some embodiments, the storage potentially provides easy accessibility of the guidewire for its reinsertion into the microcatheter.
4. Potentially keeping the guidewire away from the user and/or patient: in some embodiments, the storage potentially provides a safe location to keep the guidewire away from the user and/or patient during the surgical intervention and/or during the operation of the device.
5. Potentially manages the loose proximal end of the tool, keeping it from inadvertently reaching non-sterile areas during the procedure.

In some embodiments, the storage of the present invention provides one or more, and preferably all, of the above-mentioned potential advantages.

### Exemplary Sensing Mechanism in the Storage Compartment

In some embodiments, the storage compartment comprises one or more sensors configured to monitor the guidewire. In some embodiments, the one or more sensors monitor how much of the guidewire has left the storage compartment. In some embodiments, that information is provided to the user, which can use it to monitor and plan the procedure. In some embodiments, the one or more sensors are distributed along the storage container, and when a proximal end of the guidewire passes a specific sensor, the user is informed how much of the guidewire has left the robotic device, how much guidewire is still available to use, etc.

### Exemplary Diverter

Referring now to FIG. 4k, showing a schematic representation of an optional diverter, according to some embodiments of the invention. In some embodiments, instead or in addition to the storage compartment, the robotic device 402 comprises a diverter 450 attached to the proximal end of the robotic device 402, and configured to direct the proximal end of the guidewire when the guidewire is being moved proximally (backwards). In some embodiments, the diverter 450 is bendable and configured to remain in the bended position. In some embodiments, the diverter 450 can be bended in more than one location along the diverter 450. In some embodiments, the diverter 450 can be bended to any direction. In some embodiments, the diverter 450 may comprise any workable radius which allows, on one side, to the guidewire to freely move within the diverter 450, and on the other side, to allow connection of the diverter 450 to the robotic device 402. In some embodiments, the diverter 450 comprise a length of from about 5 cm to about 15 cm, optionally from about 3 cm to about 30 cm, optionally from about 1 cm to about 60 cm. In some embodiments, the diverter comprises a length longer than 60 cm. In some embodiments, the diverter is a 3D structure having multiple curves directing the proximal end of the guidewire through a predetermined track. In some embodiments, the diverter 450 has at least a portion that is characterized by a curved, open cross-section. In some embodiments, the diverter 450 has at least a portion that is characterized by a circular, closed cross-section.

A potential advantage of having a diverter 450 is that it potentially avoids cases where the guidewire being extracted from the proximal side disturbs the user and/or the patient. In some embodiments, directing the direction to which the guidewire will move when moved backwards provides that potential advantage.

### Exemplary Guidewire Stopper

In some embodiments, the robotic device comprises a mechanism configured to stop the proximal end of the guidewire from completely entering/exiting the robotic device, which can cause losing the control of the guidewire if completely deployed from the driving mechanism. In some embodiments, the proximal end of the guidewire is stopped at the proximal entrance in the robotic device, for example, where the storage compartment is connected. In some embodiments, the proximal end of the guidewire is stopped before exiting the robotic device at the distal entrance in the robotic device, for example, where the catheter positioned inside the patient is connected to the robotic device. In some embodiments, the mechanisms that stop the guidewire, either at the proximal entrance or at the distal entrance, are one or more of the following:
1. Mechanical hugging mechanism: in some embodiments, the robotic device comprises a hugger, workable either electronically, magnetically or pneumatically, which once activated, hugs the guidewire and stops its movement. In some embodiments, the hugging mechanism is activated either by a sensor, configured to sense the presence of the proximal end of the guidewire, or by an additional element mounted on the proximal end of the guidewire. When the additional element reaches the hugging mechanism it causes the mechanism to be activated and therefore the guidewire is stopped. Alternatively, when a distal or proximal end is detected by the sensor, a command is generated to stop the movement of the driving mechanism, thereby stopping the advancement or retraction of the guidewire.
2. Addition of a stopping element at the proximal end of the guidewire: in some embodiments, a stopping element is added at the proximal end of the guidewire. In some embodiments, the stopping element comprises a size that does not allow the stopping element to enter the robotic device. Therefore, once the stopping element reaches the robotic device, the guidewire stops. In some embodiments, the storage compartment comprises an internal size that takes under consideration the size of the stopping element. In some embodiments, the stopping element is stopped at the optional connector.

### Exemplary Methods

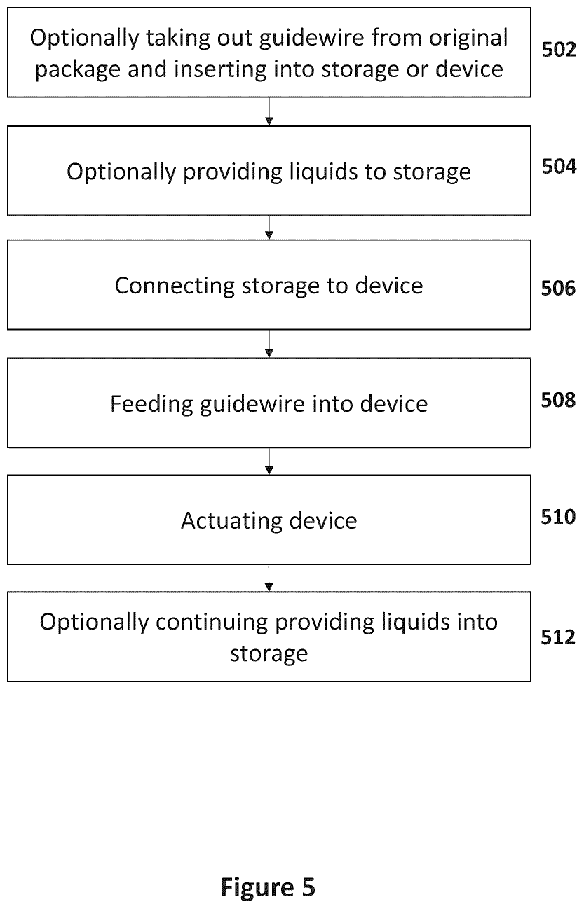

Referring now to FIG. 5 showing a flowchart of an exemplary method, according to some embodiments of the invention. In some embodiments, the method comprises one or more of the following actions:
1. Optionally taking out guidewire from original package and inserting into storage 502 or alternatively into the device and then, using the internal mechanisms of the device, into the storage. In some embodiments, when the original package of the guidewire is not used and/or configured to be attached to the device, the user will take out the guidewire from its original package and either will insert it into the storage part of the device or alternatively into the device and then, using the internal mechanisms of the device, into the storage.
2. Optionally providing liquids to storage 504. In some embodiments, the user provides liquids to the storage. In some embodiments, the storage already comprises the necessary amount of liquids to be used during the operation of the device. In some embodiments, the liquids are added to the storage or original package before connecting either to the device.
3. Connecting storage to device 506. In some embodiments, the user connects the storage to the dedicated location in the device. In some embodiments, the user connects the original package of the guidewire to the device. In some embodiments, the user connects the dedicated storage part to the device.
4. Feeding guidewire into device 508. In some embodiments, the user feeds the guidewire into the device, optionally leading with the guidewire proximal end and passing at least a portion of the guidewire through the inner pathway of the device. In some embodiments, the feeding of the guidewire onto the device is performed before the connecting of the storage to the device.
5. Actuating device 510 by inserting and/or retracting said guidewire into a microcatheter.
6. Optionally continuing providing liquids into storage 512 during said actuating.

It should be understood that the order of the actions is not fixed and that amending the order is still under the scope of the invention.

### Exemplary Tool Manipulation Assemblies

Referring now to FIG. 6a showing a schematic diagram of a driving wheel assembly for linearly and/or rotationally moving an elongate surgical tool, according to some embodiments of the invention.

In some embodiments, an elongate surgical tool 602 (e.g. a guidewire, a microcatheter) is received within a designated channel 604 of the device.

In some embodiments, a plurality of movement driving elements such as driving wheels are positioned adjacent the channel. In some embodiments, the wheels are arranged in pairs where one wheel is opposite another wheel which is located across the channel. In some embodiments, the movement assembly may contain just two wheels. In some embodiments, the movement assembly may include, for example, between 2-40 wheels, such as 10-20, 8-16, 4-30, 12-38 or intermediate, larger or smaller number of wheels.

In some embodiments, wheels on a first side of the channel (such as wheels 606, 608) are coupled to a stationary element 610 (e.g. an inner wall of the housing, a frame, a rod and the like). In some embodiments, wheels in a second opposing side of the channel (such as wheels 612, 614) are coupled to elastic or deformable elements, such as springs 616.

In some embodiments, in a rest state of the springs, wheels 612, 614 are pushed by the springs in close proximity to wheels 606, 608 such that the tool 602 is contacted by all wheels. Upon rotation of the wheels, the tool is advanced or retracted (depending on the direction of rotation of the wheels) along a long axis of the channel. In some embodiments, in the approximated state of the wheels, the opposing wheels of each pair are brought to a distance which is equal to or shorter than a diameter of tool 602, for example, equal to or shorter than a diameter of a guidewire, e.g. a guidewire diameter between, for example, 0.18-0.25 mm, 0.5-1.14 mm, 0.18-1.14 mm or intermediate, larger or smaller diameter. In some examples, when the tool is a microcatheter, the distance between the wheels is equal to or shorter than a microcatheter diameter, for example, between 2-3 FR. In some examples, when the tool is a guiding catheter, the distance between the wheels is equal to or shorter than a guiding catheter diameter, for example, between 309 FR.

In some embodiments, all wheels rotate at a similar rotational direction and speed. In some embodiments, the tool is firmly grasped between the opposing wheels, for example so that upon rotation of the assembly as a whole, the tool is caused to roll about its long axis.

In some embodiments, at a compressed state of the springs, wheels 612, 614 are retracted away from the tool, releasing hold of the tool. Optionally, a retracted position of the wheels facilitates insertion and/or removal of the tool from the channel. In some embodiments, compression of the springs is actuated via a knob or button, for example configured externally to the housing. In an example, rotation of the knob adjusts tension on the springs.

In some embodiments, the plurality of springs are actuated together as a single unit, so that all springs compress (or decompress) at once.

Referring now to FIGS. 6b-6d showing different views of a driving assembly for moving a tool, according to some embodiments of the invention.

In some embodiments, an assembly of driving wheels 620 for moving a tool 622 (e.g. a guidewire) comprises multiple adjacent pairs of opposing wheels. In some embodiments, the wheel pairs are alternatively arranged on different planes which cross each other, for example so that a first series of wheel pairs lies on a first plane 624, and a second series of wheel pairs lies on a second plane 626, and the wheel pairs of both series intervene with each other.

In some embodiments, adjacent pairs of wheels include, for example, at least two wheel pairs closely positioned relative to each other, for example such that spaces defined between opposing wheels of each the two pairs are at an axial distance of no more than 20 mm, 10 mm, 5 mm, 1 mm, 0.5 mm or intermediate, longer or shorter distance.

In some embodiments, an angle between planes 624 and 626 is between 30-120 degrees, such as 60 degrees, 90 degrees, 11 degrees, or intermediate, larger or smaller angle. In a specific example, planes 624 and 626 are perpendicular (defining a “+” shaped arrangement).

In some embodiments, tool 622 extends along an elongate channel defined by the small spaces between opposing wheels of the multiple pairs.

Some potential advantages of an assembly of driving wheels which are alternately arranged on different planes that cross each other may include effectively utilizing a volume around the tool: such arrangement provides for fitting, optionally, a large number of driving wheels in yet a relatively small volume; having the wheel pairs engage the tool at multiple locations along the length of the tool, where optionally a distance between adjacent contact locations of the wheel pairs with the tool, as measured for example along the length of the tool, is less than 6 mm, 6 mm, 5 mm or intermediate, longer or shorter distance) optionally, the distance depends on the diameter of the driving wheels used); reducing spatial interference between wheels of adjacent pairs (as each pair lies on a different plane); potentially, segments of the tool that are located in between the adjacent wheel pairs allow access to the tool (for example, to load the tool and/or remove the tool and/or manually adjust a position of tool, optionally in an emergency situation or malfunctioning).

In some embodiments, as shown for example in FIG. 6d, each of the wheels of a series on at least one side of the channel is coupled to a frame 628 which includes a spring 630 configured to advance and/or retract the wheel that is coupled to the frame upon the spring being tensioned and/or released.

In some embodiments, an elongate rod 632 passes through the plurality of frames and interfaces with them. In some embodiments, rod 632 is operably attached to a gear 634 (for example, at one of the ends of the rod) which when rotated rolls the rod, thereby changing tension on (compressing or decompressing) the springs 630. In some embodiments, two elongate rods (of the two series, each arranged on a different plane) are rolled simultaneously, for example by rotation of a knob gear 636 which is coupled to the gears 634 of both rods, so that when knob gear 636 is rotated, gears 634 rotate as well, rolling rods 632 to change tension on the springs, which thereby retract the wheels from the channel or advance the wheels towards the channel. A potential advantage of an arrangement of rods and their actuating gears for example as shown in FIG. 6d may include simultaneously setting a position of the wheels of both series (relative to the channel) via a single component, for example by rotation of the knob gear.

In some embodiments, rotation of driving wheels 620 is actuated by a motor (not shown). Optionally, a plurality of transmission gears (not shown) transfer torque from the motor to the driving wheels.

In some embodiments, each series of wheels (i.e. wheel pairs lying on a single plane) includes for example between 2-16 wheels, for example arranged as 1-8 pairs. In such construction, the full assembly (including the two series) includes for example between 4-32 wheels in total, arranged for example as 2-16 pairs.

In some embodiments, the total number of wheels is selected so that sufficient traction is provided, for example by having a large enough number of contact locations between the tool and each of the wheels. Some potential advantages of multiple contact locations between the tool and each of the wheels may include: reducing a risk of slippage of the tool, improved grasping of the tool (such as in between the opposing wheels of each pair), and an ability of using of the wheels as pinching elements of the guidewire during rotation, allowing to reduce the individual pinching forces applied by each wheel pair onto the tool for gaining a same total grasping force of the tool with less impact on the tool surface.

It is noted that additional driving wheel arrangements are contemplated herewith. In some embodiments, wheel pairs may be arranged to lie on multiple planes, for example, on more than two planes. In an example, wheel pairs are helically arranged about the long axis in a spiral configuration.

In some embodiments, the channel defined by the plurality of spaces between opposing wheels of adjacent pairs is a linear, straight channel. Alternatively, the channel comprises one or more curvatures.

Referring now to FIGS. 6e-6f showing a construct for linearly moving and/or rotating a tool, according to some embodiments of the invention.

FIGS. 6e-f are views from two different angles of a construct 638 configured to receive a tool (e.g. a guidewire), according to some embodiments. In some embodiments, the construct is housed within in an upper portion of a robotic device for example as described herein.

In some embodiments, the construct includes an assembly of driving wheels, for example as described in FIGS. 6e-g, which are positioned to contact a tool that is passed through the construct (such as passed in its designated channel, in-between the driving wheels and along the length of the construct). In some embodiments, the driving wheels assembly is substantially centered with respect to the whole construct.

In some embodiments, the construct comprises one or more motors, for example a motor 640 configured to actuate rotation of the driving wheels. In some embodiments, motor 640 is configured to rotate with the construct as a single unit when the construct is rotated. Optionally, motor 640 is axially aligned with the construct.

In some embodiments, another motor 642 is configured to actuate rotation of the construct as a whole, including rotation of motor 640 and motor 642 themselves. In some embodiments, motor 642 is positioned within a space formed in the construct. In some embodiments, motor 642 is configured to be positioned within this space, without extending beyond the perimeter defined by the construct's edges.

In some embodiments, a plurality of transmission gears 644 transfer torque from motor 642 to a large gear wheel 646 which when rotated rotates the construct as a whole.

In some embodiments, the construct comprises a plurality of transmission gears 648, which transfer torque from motor 640 to the driving wheels which linearly move the tool. Optionally, the transmission gears are located radially externally to the driving wheels. Optionally, rotation of each driving wheel is driven by one or more transmission gears. In some embodiments, a number and/or shape and/or position and/or size of the transmission gears is selected to modify the speed of rotation dictated by the motor. For example, the transmission gears reduce the speed of the motor. In some embodiments, all driving wheels are driven by the transmission gears at a similar speed. In some embodiments, at least 2, 4, 10, 14, 16, 20 or intermediate, larger or smaller number of transmission gears are positioned and configured to drive movement of each of the pairs of driving wheels.

In some embodiments, the construct is coupled to slip ring 650 through which electrical power may be supplied to the one or more motors. In some embodiments, slip ring 650 is configured to ensure electrical contact at all rotational orientations of the construct. In some embodiments, slip ring 650 is axially aligned with the construct.

In some embodiments, in use, linear movement (advancement and retraction) of a tool received within the construct is carried out in the following manner: motor 640 drives rotation of transmission gears 648, which in turn optionally adjust the speed of rotation and transfer torque from the motor to the driving wheels (not shown), which are in held in close contact with the tool. In some embodiments, roll movement of the tool is carried out by rotation of gear 646 operated by motor 642 via transmission 644, where gear 646 rotates the construct as a whole, causing the tool held by the driving wheels to roll about its long axis.

As can be further observed, in some embodiments, a knob 652 which is optionally external to the device housing drives simultaneous rotation of gears 654, each of which is coupled to an elongate rod (not shown). When each of the rods is rotated, it changes the tension applied onto a plurality of springs (not shown) to either approximate or pull away each of the driving wheels that are coupled to the springs from their opposing driving wheels.

In some embodiments, construct 638 is compactly arranged so that its components are maintained within a limited radial extent, for example, a radius at a cross section of a substantially cylindrical construct is less than 3.5 cm. Optionally, a volume of the construct is smaller than 500 cm\hat{ }3.

In some embodiments, components forming the construct are co-centrically arranged about a similar long axis. In some embodiments, gear 646 and/or slip ring 650 are arranged to lie on planes that are substantially perpendicular to a long axis of the construct, and do not protrude more than 5%, more than 10%, more than 15% or intermediate, larger or smaller percentage beyond a perimeter defined by the construct. A potential advantage of a compact co-centrical arrangement of the components of the construct may include maintaining a relatively short radius of rotation of the construct, when rotated as a single unit (such as to generate roll of the tool).

Referring now to FIGS. 6g-6i showing an assembly for linearly moving a tool, according to some embodiments.

In some embodiments, assembly 656 is configured for linearly advancing and/or retracting a tool 658, such as a guidewire, microcatheter or guiding catheter.

In some embodiments, the assembly comprises a plurality of driving wheels 660, optionally arranged in two parallel rows, such that each pair of opposing wheels defines a path therebetween for receiving the tool.

In some embodiments, wheels of at least one of the rows of the assembly are coupled to elastic elements such as springs 662 which move the wheels towards or away from tool 658 upon change in tension. In some embodiments, each driving wheel is coupled to a spring. Alternatively, a plurality of driving wheels of a row, and optionally, all driving wheels of a row are coupled to a same spring (such as via a connecting frame or rod, not shown). In some embodiments, spring 662 is encased within a compartment 664. In some embodiments, tension on the springs is modified via rotation of knob 666 which in turn rotates a rod 668 that extends axially across all compartments 664, and pulls or compresses the springs once rotated.

In some embodiments, a plurality of transmission gears 670 are positioned in operable contact with the driving wheels 660 and are configured to transfer torque from a motor (not shown) and/or to adjust the actuation speed dictated by the motor.

In some embodiments, at an entry and/or exit location of the channel in which the tool passes, a seal 672 is provided. In some embodiments, the seal includes an aperture which can be pushed open by advancement of the tool through. In some embodiments, the seal hermetically surrounds the tool around the aperture, preventing fluid (e.g. saline, blood, water) from flowing into the channel between the driving wheels.

### Methods for Detecting and Positioning a Tool

In some embodiments, as mentioned above, the driving assembly drives the movement of the elongate surgical tool, the driving assembly comprising a plurality of pairs of driving elements such as driving wheels where at least one of the pairs lies on a plane different than at least one other plane on which a different, optionally adjacent wheel pair lies. In some embodiments, the wheel pairs are arranged on at least a first plane and a second plane, the first plane crossing the second plane. Optionally, the first and second planes are perpendicular to each other. Optionally, the wheels pairs are interveningly disposed such that a first pair of wheels lies on the first plane, a second pair of wheels lies on a second plane, a third pair of wheels lies on the first plane, a fourth pair of wheels lies on the second plane, and so forth. In some embodiments, each pair of wheels defines a space therebetween, and the plurality of wheels pairs are arranged about a similar long axis so that the plurality of spaces form an elongate channel for receipt of an elongate surgical tool (e.g. a guidewire). When the elongate surgical tool is received within the channel, the driving wheels contact the tool at a plurality of locations along the length of the tool, so that when the driving wheels are rotated, the tool is caused to move linearly (e.g. be advanced or retracted along the channel). In some embodiments, a driving assembly acts as a manipulator of the tool, and is configured to move the tool, for example, advance and/or retract the tool, roll the tool.

In some embodiments, additionally, the device allows controlling linear movement of the tool in a designated channel of the robotic device by setting a reference location for the tool along the channel. In some embodiments, using one or more sensors such as optic sensors positioned along the channel, a tool position is monitored at least twice: once when the tool reaches (for example, is advanced to) a position in which its presence is detected by the sensors, and once when the tool is moved (e.g. retracted or advanced) to a second position in which its presence is no longer detectable by the sensors. In some embodiments, the second position is used a reference location, for example according to which additional movements of the tool, optionally, automated movements, are carried out, using the reference location for calibration purposes. In some embodiments, a number of motor rotations required for moving the tool between the first and second positions is counted, e.g. via an encoder, and that number is used for further control of the tool, for example when automatically retracting or advancing the tool between the two positions.

In some embodiments, as mentioned above, retraction and advancement of tools, such as guidewires, are steps that may be performed many times during a single procedure. For example, when contrast agent is to be injected through the lumen of a microcatheter, the guidewire is first retracted from the microcatheter lumen to allow room for the contract agent to pass through, and once the injection is complete, the guidewire may be re-introduced into the microcatheter for continuing the procedure. A potential advantage of automatic retraction and/or advancing of the tool between two positions may include reducing the amount of time required for these actions, for example as compared to manual advancement/retraction. Automated movement of a tool, such as between two defined positions or along a predetermined distance relative to one of the two positions, may potentially save time for the overall procedure and specifically time of exposure to radiation (such as due to imaging being carried out simultaneously). In such cases, as opposed to manual retraction and advancement, automation keeps the tools set in place and/or accurately transfers the tools between positions. Therefore, using automation can be done very fast, and substantially without risk of unwanted movements, for example as compared to manually operated movement.

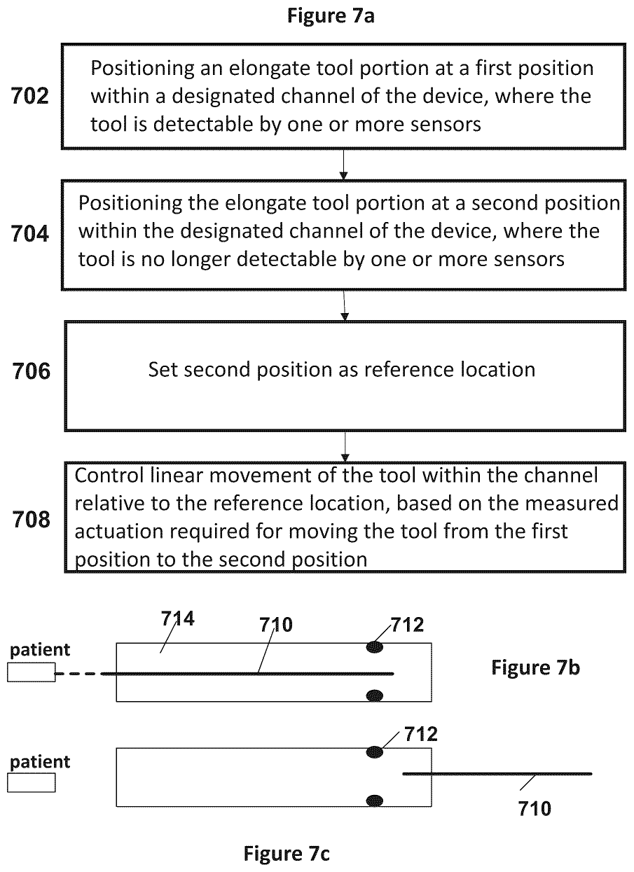

Referring now to FIG. 7a showing a flowchart of a method for setting a reference position of an elongate tool in its designated channel of the robotic device, according to some embodiments.

In some embodiments, the robotic device comprises one or more sensors, for example sensors configured to detect presence and/or a relative position of a tool loaded onto the device. In some embodiments, the one or more sensors are positioned along the channel in which the tool is received. Optionally, a plurality of sensors (e.g. optic sensors) are positioned at a plurality of axial positions along the channel and/or at a plurality of circumferential positions of the channel. In some embodiments, for example in the case of a channel that is large in volume, different sensors may be provided for covering different portions of the total volume of the channel.

In some embodiments, sensing of presence of a tool is performed for calibration purposes, for example to set a reference axial position for the tool relative to the long axis of the channel.

In some embodiments, at 702, an elongate tool is introduced into its channel and advanced (manually and/or automatically) into a first axial position in the channel, in which presence of the tool (or a selected portion of it, such as a distal end or a proximal end of the tool) is detected by one or more sensors of the channel.

Then, at 704, the tool is advanced or retracted to a second position in which the tool is no longer detected by the sensor(s). In some embodiments, the number of motor rotations required to move the tool from the first position to the second is counted, e.g. by an encoder of the motor.

At 706, the second position is set as a reference location for the tool, so that at 708, linear movement of the tool within the channel can be controlled using the second position as reference, based on the measured actuation required for moving the tool from the first position to the second position, e.g. based on the counted number of motor rotations.

In some embodiments, during manipulation of the tool, using the counted number of motor rotations required for moving the tool from the first position to the second, quick retraction or advancement of the tool from or to the reference location may be carried out by automated activation of the motor to rotate the counted number of rotations. A potential advantage of automated retraction and/or advancement of the tool which is carried out, for example, by commanding the motor to rotate the counted number of rotations, may include faster movement of the tool for example as compared to manually controlled advancement or retraction.

In some embodiments, automatic advancement of the tool is performed from the second position to a third position, the third position being located proximally to the first position and distally to the second position. Optionally, in order to bring the distal end of the tool to the third position, the tool is advanced automatically by commanding the motor to rotate less than the counted number of rotations, for example, by reducing a predetermined number from the count of rotations that were required to move the tool between the first and the second positions. The distance between the first position and the third position can be, for example, 1, 2, 3, 4, 6, 8, or 10 cm, or intermediate, longer or shorter distance. A potential advantage of returning the tool to a third position being proximal relative to the original first position lies in safety considerations. For example, the tool is automatically advanced back into the patient at a relatively high speed up to the third position, but the physician controls the speed and amount of advancement beyond the third position, which is closer to the point of interest and may include a more sensitive environment.

In some embodiments, tool advancement and retraction are performed at a position of an optional connector. In an example, a guidewire is retracted to allow injection of fluid (e.g. contrast agent) into the lumen of a microcatheter, and following injection, the guidewire is advanced once again into the microcatheter lumen. In such exemplary configuration, automated retraction and advancement of the tool may accelerate and facilitate the injection process.

Referring now to FIG. 7b schematically showing a tool 710 in the first position, being detected by one or more sensors 712 of a channel 714; FIG. 7c schematically shows the tool 710 in the second position, where it is no longer detected by the one or more sensors 712.

As used herein with reference to quantity or value, the term “about” means “within±20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc. ; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A storage for elongated surgical tools used in a robotic device, the storage comprising a hollowed body sized and shaped to store at least one elongated surgical tool within; said body comprising a proximal end configured to be connected to said robotic device; said connecting allows an internal space of said storage to be in communication to an internal pathway of said robotic device, thereby allowing said elongated surgical tool stored in said storage to be driven back and forth between said storage and said robotic device.

2. The storage according to claim 1, further comprising a connector for reversibly connecting said hollowed body to said robotic device; and wherein said connector is either an integral part of said storage or is provided with said storage and said elongated surgical device.

3-4. (canceled)

5. The storage according to claim 1, wherein said hollowed body comprises an internal diameter sized and shaped to store one or more liquids in addition to said elongated surgical tool.

6-7. (canceled)

8. The storage according to claim 1, wherein said hollowed body is configured to store said elongated surgical tool while avoiding bending and/or damaging said elongated surgical tool.

9. The storage according to claim 2, wherein said connector comprises one or more connecting elements configured to allow connection of storages having different external diameters to said robotic device; and wherein said one or more connecting elements comprise one or more concentric steps narrowing in diameter along an inner wall of said connector.

10-12. (canceled)

13. The storage according to claim 1, wherein said storage comprises a sack/bag.

14. The storage according to claim 1, wherein said storage comprises one or more of:
a. a reel for reeling in and out said elongated surgical tool from said robotic device;
b. a mechanical support for said storage; wherein said mechanical support is either part of said storage or part of said robotic device;
c. a bridging element configured to provide geometrical alignment between said proximal end of said storage and said robotic device.

15-18. (canceled)

19. The storage according to claim 1, wherein said hollowed body is as long as said elongated surgical tool.

20. The storage according to claim 1, wherein said body is configured to gravitationally hang from said robotic device and stiff enough to prevent damage and/or allow free movement of said elongated surgical tool.

21. The storage according to claim 5, wherein said one or more liquids are one or more of saline, water, oil and lubricant.

22. A method of using a storage for an elongated surgical tool used in a robotic device, the method comprising:
a. connecting said storage to said robotic device; an internal space of said storage to be in communication to an internal pathway of said robotic device;
b. feeding an end of said elongated surgical tool into said robotic device;
c. actuating said surgical device to move said elongated surgical tool;
d. driving said elongated surgical tool back and forth from said storage to said robotic device and vice versa.

23. (canceled)

24. The method according to claim 22, further comprising automatically retracting said elongated surgical tool from a location inside a second elongated surgical tool into said storage.

25. The method according to claim 24, automatically returning to said location inside said second elongated surgical tool.

26. The method according to claim 22, before said connecting, the method further comprises taking out said elongated surgical tool from its original package and inserting said elongated surgical tool into said storage or taking out said elongated surgical tool from its original package and inserting said elongated surgical tool into said robotic device before inserting said elongated surgical tool into said storage.

27. (canceled)

28. The method according to claim 22, wherein said connecting comprises connecting an original package of said elongated surgical tool to said robotic device and utilizing said original package as said storage.

29. The method according to claim 22, further comprising providing one or more liquids to said storage, and further comprising continuing providing liquids into said storage during said actuating.

30. The method according to claim 22, further comprising maintaining one or more of humidity, cleanness and sterility of said elongated surgical device by said storage.

31. The method according to claim 22, further comprising keeping said elongated surgical tool wet during said actuating of said surgical device.

32. The method according to claim 22, wherein said feeding is performed before said connecting.

33-34. (canceled)

35. A system for manipulation of at least one elongated surgical tool, comprising:
a. a robotic device configured to manipulate said at least one elongated surgical tool;
b. a storage according to claim 1.

36-43. (canceled)
